(19) United States
(12) Patent Application Publication
(10) Pub. No.: US 2011/0229852 A1
(43) Pub. Date: Sep. 22, 2011

(54) MEDICATED DENTAL IMPLANT

(75) Inventors: Alfonso Petrosino, Vedano Olona (Varese) (IT); Raffaele Stoppano, Cogoleto (Genova) (IT)

(73) Assignee: GIESSE TECHNOLOGY S.R.L., Cogoletto (Genova) (IT)

(21) Appl. No.: 12/671,956

(22) PCT Filed: Jul. 29, 2008
(86) PCT No.: PCT/IB08/53040

§ 371 Date: Apr. 30, 2010

(30) Foreign Application Priority Data
Aug. 3, 2007 (IT) MI2007A001617

(51) Int. Cl. A61C 8/00; A61B 19/02

(52) U.S. Cl. 433/174; 206/63.5

(57) ABSTRACT

A dental implant is disclosed of the type having a threaded pin and a temporary closing screw (30) which has a shape capable of retaining, between the closing screw and the upper surface of the threaded pin, an insert of biologically inert material (31) embedding, in a slow-release manner, a pharmacological agent capable of promoting the grafting of the implant.

### FIELD OF THE INVENTION

The present invention concerns an improved dental implant.

### BACKGROUND ART

As known, dental implantology is a surgical technique which allows to implant a dental prosthesis into a patient's jawbone.

The surgical technique provides to incise the mucous membrane, create a series of seats in the patient's gum bone by means of a calibrated osteotribe, and then to implant threaded screw pins which represent the fastening member of the actual dental prostheses.

A complete and finished exemplary implant member is illustrated in FIG. 1. Here a threaded pin 1 is disclosed, securely implanted in bone tissue T, wherefrom a stub 2 projects—securely screwed in the inner seat of pin 1—whereon an artificial tooth D is mounted. It must be noted that pin 1 is intended to be fully implanted in the bone tissue, while gum G closes up above pin 1 and around stub 2.

The final definition of the plant, however, is arrived at with a certain gradualness. As a matter of fact, after the first implant stage, the threaded pin is left inoperative in the bone for it to graft adequately, before being able to mount the actual dental prosthesis on top of said pin. In this time interval, which may last up to three-four months, gum cicatrisation and neoformation of bone tissue takes place, which then allow the subsequent operation of application of the fixed prosthesis. The clinical method, in this transitory stage, provides for the prosthesis-fixing seat—which is provided in the threaded pin—to be closed by so-called covering screws.

The covering screws are normally intended to close a hole found in the pin, while the gum is sutured thereabove and cicatrises.

FIGS. 2 and 3 show a pin 1 and a covering screw 3 belonging to the prior art, respectively.

The covering screws suitable for this purpose generally consist of a shaft or stem 3a, partly or entirely threaded, and of a screw head 3b, which may be of a varying thickness.

At the top of the screw head a maneuvering seat is provided, such as an Allen-key hole, for the introduction of a tool which enables the surgeon to tighten the closing screw in the corresponding threaded seat of pin 1 (not shown in FIG. 2).

For such purpose, according to the prior art pin 1 may have a simple top abutment surface 1a—against which the lower portion of screw head 3b abuts—or a proper widened collar (not shown). The collar also acts as a valid end stop of pin 1 on the outer surface of the bone.

It has been detected that this transitory implant stage is often a critical moment for the development of side effects, related to the incision of the bone tissue and of the soft tissues, such as for example inflammations and infections of the structures around the implant. Similar side effects may cause pain conditions which are annoying to the patient, as well as a slowing down or failure of implant grafting.

### SUMMARY OF THE INVENTION

The object of the present invention is hence to supply an improved implant which is capable of alleviating the cited problems, allowing a quick and trouble-free post-surgery progress, with effective implant graft and no gum infections or inflammations arising.

Said object is achieved by an implant having the features described in the attached main claim, the dependent claims relating to some particularly preferred features.

### BRIEF DESCRIPTION OF THE DRAWINGS

The implant according to the invention will now be described in detail with reference to the accompanying drawings, wherein

FIG. 1 is a section view of a prior-art implant;

FIG. 2 is a side elevation view of a prior-art pin;

FIG. 3 is a perspective view of a prior-art closing screw;

FIG. 4 is a side elevation view of an implant according to a first embodiment of the invention; and

FIG. 5 is a side elevation view of an implant according to a second embodiment of the invention.

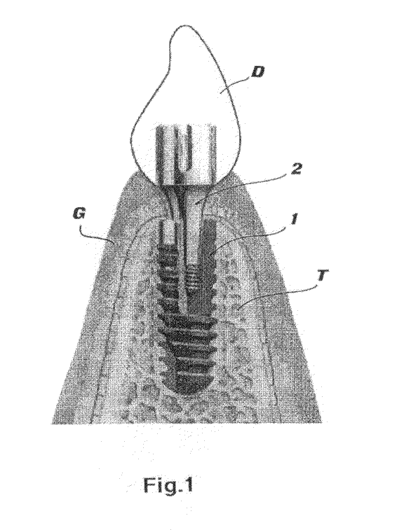

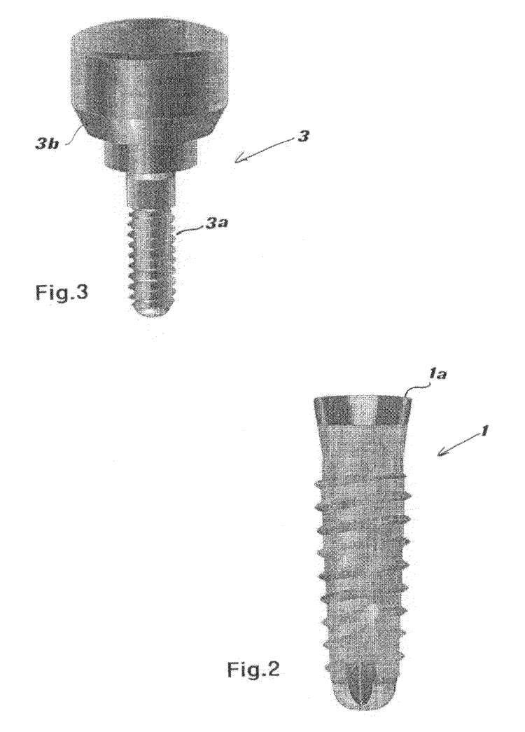

### DETAILED DESCRIPTION OF PREFERRED EMBODIMENTS

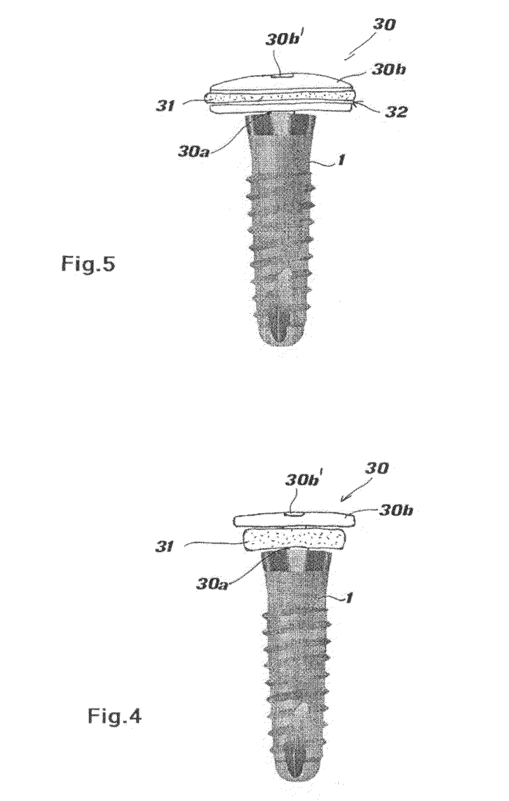

FIG. 4 shows an implant according to a first embodiment of the invention. A threaded pin 1 has a shape known per se. Pin 1 may be manufactured in various sizes and is generally made of biocompatible metallic material, such as titanium.

Above pin 1 a closing screw 30 is provided to be coupled, at least during the transient phase of bone graft, said screw consisting of a threaded screw stem 30a (partly visible in FIG. 4) and of a proper head 30b, said head being provided with a seat 30b′ for the engagement with a tightening tool.

According to the invention, between threaded portion 30a and head 30b, an annular body 31 of soft and biocompatible material is provided, wherein a slow-release drug, in particular an antibiotic, is dispersed.

The drug is selected among those best suited to promote a positive outcome of the graft of pin 1 in the bone tissue and to prevent the proliferation of infections and inflammations. Therefore, a particularly advantageous drug may be a drug of the antibiotics family or a biphosphonate, which promotes bone growth.

Ring 31 consists of a soft material suited to embed a drug which is then slowly released. For such purpose a silicon-rubber-based material is particularly well-suited, of the type already employed in the medical tool field in catheters for pacemaker apparatuses.

For example, a suitable silicone is the one manufactured by Dow Corning Corporation or by NuSil Technology LLC or by Applied Silicone Corporation.

In view of the use of such material, available powder antibiotics prove to be preferable, since they may be easily mixed in an industrial process with the silicone of which ring 31 is made. Such an antibiotic is for example powder metronidazole.

Other materials which may be employed for the building of the drug-enriched ring may be other basic polymers, such as polyurethane, polyurethane copolymer, fluoropolymer or a polyolefin. In such case, the drug or biologically-active agent may create covalent bonds on the basic polymer, such as terminal groups, or in the basic polymer, attaching to the backbone.

Ring 31 is preferably moulded into a toroidal shape, with the outer diameter substantially equivalent to that of head 30.

The height and width of the ring in the last analysis determine the volume thereof and hence the drug-containing ability thereof. These sizes will hence be chosen depending on the specific application and on the desired drug release speed or on the absorbance ability thereof by the patient.

Ideally, an adequate release speed for a drug is the one which allows to achieve pharmacologic agent activity in the interval comprised between two-three days up to three months from implantation.

In order to avoid undesired squashing of ring 31 during tightening of screw 30, the threaded shaft 30a of the closing screw has a shoulder (not visible) intended to abut with the outer surface of pin 1, so as to establish a tightening end stop.

Alternatively, it is possible to provide a spacing sleeve (not shown), to be inserted onto the threaded shaft before applying medicated ring 31 and the subsequent coupling with pin 1.

In the package supplied to the dentist, together with an implant pin 1, a plurality (for example three) of different-size closing screws and a plurality of different-length spacing sleeves are hence supplied, so as to adjust the implant to the specific application.

According to a second embodiment of the invention (FIG. 5), ring 31 is arranged within a slot or groove 32 provided in head 30.

The implant described here allows to perfectly achieve the desired objects. As a matter of fact, the implant of the invention is provided with a closing screw suitable to retain a cap or a ring enriched with a pharmaceutical substance which promotes a swift grafting of the implant, without generating side-effects. The achievable result is particularly effective should the closing screw be intended to remain fully below the gum, because it avoids the formation of pathogenic bacteria, which are particularly aggressive in these conditions.

Finally, the localised administering of the drug frees the physician from administering the same drug systemically, with evident advantages in terms of side effects and appeal to the patient.

It is understood, however, that the invention is not limited to the specific configurations illustrated above, which represent only non-limiting examples of the scope of the invention, but that a number of changes may be made without departing from the scope of the invention, which is instead defined by the attached claims.

For example, although a closing screw has been illustrated which has an evident wide head 30, it is easily understandable that the invention extends also to the case wherein the screw has a smaller head or has no head at all: in this last case, the screw would consist only of a threaded stem 30a, in the upper portion whereof there is arranged a seat for the action of a tightening tool and a suitable housing for the engagement with ring 31 or an equivalent closing cap of the implant acting as a drug carrier.

Finally, although reference has always been made to a drug-enriched ring, which represents by all means the most preferred form, it is not ruled out that a similar, different-shape insert may be provided to be engaged with a corresponding seat defined by the temporary portion of the implant.

1) Dental implant of the type comprising a threaded pin (1) and a temporary closing screw (30), characterised in that it has a shape capable of retaining, between said closing screw (30) and the upper surface of the threaded pin (1), an insert (31) of biologically inert material embedding, in a slow-release manner, a pharmacological agent capable of promoting the grafting of the implant.

2) Implant as claimed in claim 1, wherein said insert (31) is a toroidal ring.

3) Implant as claimed in claim 2, wherein said closing screw (30) has a circular groove (32) wherein said ring (31) is housed.

4) Implant as claimed in claim 2, wherein said closing screw (30) has a head (30b) and a shaft (30a) for engagement with pin (1) and said ring (31) is on said shaft (30a) between the head (30b) and the pin (1).

5) Implant as claimed in claim 1, wherein said ring (31) is made of a soft polymeric material.

6) Implant as claimed in claim 5, wherein the base of said ring (31) is silicone rubber.

7) Implant as claimed in claim 1, wherein said pharmacological agent contains at least one antibiotic.

8) Implant as claimed in claim 1, wherein said pharmacological agent contains biphosphonates.

9) Temporary closing screw for a dental implant as claimed in claim 1, characterised in that it has a circular groove (32) wherein there is engaged a ring (31) of biologically inert material embedding, in a slow-release manner, a pharmacological agent capable of promoting the grafting of the implant.

10) Surgical package for a dental implant, characterised in that it comprises an implant as claimed in claim 1 and a plurality of different-height, temporary closing screws.
